(19) United States
(12) Patent Application Publication
(10) Pub. No.: US 2021/0055288 A1
(43) Pub. Date: Feb. 25, 2021

(54) IMMUNOCHROMATOGRAPHIC TEST STRIP FOR DETECTING DIGOXIN AND USES THEREOF

(71) Applicant: Beijing Diagreat Biotechnologies Co., Ltd., Beijing (CN)

(72) Inventors: Jianping ZHOU, Beijing (CN); Yujun ZHOU, Beijing (CN); Xiuli XU, Beijing (CN)

(73) Assignee: Beijing Diagreat Biotechnologies Co., Ltd., Beijing (CN)

(21) Appl. No.: 16/598,834

(22) Filed: Oct. 10, 2019

(30) Foreign Application Priority Data
Aug. 23, 2019 (CN) 201910782799.7

(51) Int. Cl. G01N 33/543; G01N 33/58; G01N 33/533

(52) U.S. Cl. CPC G01N 33/54306; G01N 33/9453; G01N 33/533; G01N 33/582

(57) ABSTRACT

The invention provides immunochromatographic test strips for detecting digoxin and uses thereof. The immunochromatographic test strips include a bottom plate, a sample pad, a glass fiber mat, a nitrocellulose membrane and a water absorbing paper that are sequentially overlapped on the bottom plate. The glass fiber mat is sprayed with a digoxin-specific antibody-fluorescent microsphere complex. The nitrocellulose membrane is sequentially provided with a detection line coated with digoxin protein conjugate and a quality control line coated with a secondary antibody. The digoxin-specific antibody-fluorescent microsphere complex is obtained by conjugation between a digoxin monoclonal antibody or a digoxin polyclonal antibody and a fluorescent microsphere. The digoxin protein conjugate includes a digoxin-conjugated rabbit albumin or a digoxin-conjugated rabbit ovalbumin. The test strips provided by the invention are low cost, provide simple and rapid use, and have excellent stability and high sensitivity.

### FIELD OF THE INVENTION

The invention relates to the field of immunodetection technology, and in particular to an immunochromatographic test strip for detecting digoxin and uses thereof.

### BACKGROUND OF THE INVENTION

Digoxin is a medium-effect cardiac glycoside drug. During treatment, its effect on the heart is presented as a positive inotropic effect, which slows heart rate and inhibits cardiac conduction. It is suitable for treating low-output congestive heart-failure, atrial fibrillation, atrial flutter, and paroxysmal supraventricular tachycardia. Among digitalis drugs, digoxin is excreted fastest with the least accumulation; and oral absorption of digoxin is incomplete and irregular. In the “2018 Guidelines for the Diagnosis and Treatment of Heart Failure in China,” it was recommended that adverse reactions, such as digoxin toxicity, should be strictly monitored and the drug concentration should be maintained at 0.5-0.9 ng/mL. 0.8-2 ng/ml is generally used as an effective therapeutic concentration range. There is a huge variation in the pharmacokinetics of digoxin among individuals, which is influenced by both in vitro and in vivo factors. Therefore, monitoring of the drug concentration in a patient treated with digoxin is of great significance for clinical safety, rational drug use, and individualized treatment.

With the initiative of precision medicine and personalized medicine, detection of various drug concentrations is receiving more and more attention. Presently, commonly used methods for detecting digoxin are mainly mass spectrometry, high-performance liquid chromatography, immunoturbidimetry, chemiluminescence, and enzyme multiplied immunization. Mass spectrometry is a method in which electric and magnetic fields are used to separate moving ions according to their mass-to-charge ratios; and then detection of the ions is conducted, e.g., using a mass spectrometer. High-performance liquid chromatography uses a liquid as a mobile phase and adopts a high pressure infusion system to pump the mobile phase, such as a single solvent having different polarities, mixed solvents of different proportions, or a buffer solution, into a chromatographic column filled with a stationary phase. After separation, the components in the column enter a detector for detection, thus realizing analysis of the sample. The analysis instrument is a high-performance liquid chromatograph instrument. The results of chromatography and mass spectrometry are accurate, but sample preparation is required before sample loading, the detection time is long, and only one sample can be loaded at a time, so that high-throughput cannot be achieved. The cost of the detection instrument is also high, which limits their use in clinical situations. Immunoturbidimetry is a method that is mainly used by F. Hoffmann-La Roche Ltd. (Roche) for detection, and requires a matching high-cost biochemical analyzer and refrigerated storage of the reagents, which cannot meet the increasing clinical detection demands and is not able to achieve detection for a single person at any time. Chemiluminescence is a method that is mainly used by Abbott Laboratories (Abbott) and Roche for detection. For this method, it is necessary to provide a high-cost chemiluminescence instrument, to store the reagents under refrigeration, and to provide a package of 100 servings/kit. Some small hospitals cannot provide such a high sample size, resulting in a waste of reagents or expiration of the reagents before they can be used. Chemiluminescence methods are also disclosed in Chinese Patent Application No. 201711217116.0 to Zecen Biotech Co., Ltd. (Taizhou). The main manufacturer using enzyme multiplied immunization methods to detect digoxin is Siemens AG. In using the immunization method, multiple samples can be simultaneously determined on an automatic biochemical analyzer to achieve high-throughput rapid determination. The method requires the use of an automatic biochemical analyzer, and storage temperature of 2-8° C., requiring cold-chain transportation. Such methods cannot be made into a single-use package, and cannot provide timely monitoring in a medication department.

### SUMMARY OF THE INVENTION

In view of the above, an objective of the invention is to provide immunochromatographic test strips for detecting digoxin and uses thereof. The immunochromatographic test strips provided by the invention have a wide sample type, excellent stability, and high sensitivity.

In order to achieve the foregoing objective, the invention provides an immunochromatographic test strip for detecting digoxin, which includes a bottom plate, and a sample pad, a glass fiber mat, a nitrocellulose membrane and a water absorbing paper which are sequentially overlapped on the bottom plate, where the glass fiber mat is sprayed with a digoxin-specific antibody-fluorescent microsphere complex; and the nitrocellulose membrane is sequentially provided with a detection line coated with digoxin protein conjugate and a quality control line coated with a secondary antibody;

the digoxin-specific antibody-fluorescent microsphere complex is obtained by conjugation between a digoxin monoclonal antibody or a digoxin polyclonal antibody and a fluorescent microsphere;

the digoxin protein conjugate includes a digoxin-conjugated rabbit albumin or a digoxin-conjugated rabbit ovalbumin.

Preferably, the mass percentage concentration of the digoxin-specific antibody-fluorescent microsphere complex is 0.01-0.1%; and the spraying amount of the digoxin-specific antibody-fluorescent microsphere complex on the glass fiber mat is 1-10 μl/cm.

Preferably, the concentration of the digoxin protein conjugate is 1-3 mg/mL; and the spraying amount of the digoxin protein conjugate on the nitrocellulose membrane is 1-5 μl/cm.

Preferably, the secondary antibody includes a goat anti-mouse IgG antibody.

Preferably, the concentration of the goat anti-mouse IgG antibody is 1-3 mg/mL.

The invention further provides a use of immunochromatographic test strips described above in preparing a kit for detecting the digoxin content in a sample.

Preferably, the detecting of the digoxin content in the sample includes the steps of:

1) adding a sample to be tested dropwise onto a sample pad for chromatography;

2) reading fluorescence signal values, i.e., a T value and a C value, for the detection line and the quality control line of the immunochromatographic test strip after the chromatography; and

3) substituting the fluorescence signal values T/C into a standard linear regression equation y=ax³+bx²+cx+d to calculate the digoxin content in the sample to be tested.

Preferably, the test sample includes serum, plasma, or whole blood.

Compared with the prior art, the immunochromatographic test strip for detecting digoxin and the use thereof provided by the invention have the following advantages:

1) as compared with mass spectrometry and high-performance liquid chromatography, the immunochromatographic test strips of the invention have low detection cost, avoids complicated pre-processing processing, allows for quick detection, can realize high-throughput detection, is packaged for single use, and has excellent stability and high repeatability; and

2) as compared with turbidimetry and chemiluminescence, the immunochromatographic test strips of the invention have low detection cost, can be stored at room temperature, is packaged for single use, can use either serum, plasma, or whole blood sample, and have excellent stability and high sensitivity.

### BRIEF DESCRIPTION OF THE DRAWING

FIG. 1 is a diagram showing a calibration curve of digoxin; and

FIG. 2 is a diagram showing the correlation of a digoxin detection reagent

### DETAILED DESCRIPTION OF THE PREFERRED EMBODIMENT

The invention provides an immunochromatographic test strip for detecting digoxin, which includes a bottom plate, a sample pad, a glass fiber mat, a nitrocellulose membrane, and a water absorbing paper that are sequentially overlapped on the bottom plate; the glass fiber mat is sprayed with a digoxin-specific antibody-fluorescent microsphere complex; and the nitrocellulose membrane is sequentially provided with a detection line coated with digoxin protein conjugate and a quality control line coated with a secondary antibody;

the digoxin-specific antibody-fluorescent microsphere complex is obtained by conjugation between a digoxin monoclonal antibody or a digoxin polyclonal antibody and a fluorescent microsphere; and

the digoxin protein conjugate includes a digoxin-conjugated rabbit albumin or a digoxin-conjugated rabbit ovalbumin.

The bottom plate can be of any type as known by one of ordinary skill in the art, and a bottom plate used by a conventional test strip can be used.

The sample pad can be of any material as known by one of ordinary skill in the art. Preferably the sample pad includes a glass fiber, a polyester film, a cellulose filter paper, a nonwoven fabric, or combinations thereof. In the invention, the sample pad is preferably subjected to an immersion treatment; and the chemical for the immersion treatment is preferably a Triton X-100 nonionic surfactant.

The glass fiber mat can be any glass fiber mat as known by one of ordinary skill in the art, and a commercially available product of a conventionally-prepared test strip can be used. In the invention, the glass fiber mat acts as a conjugate pad. The glass fiber mat of the invention is preferably sprayed with a digoxin-specific antibody-fluorescent microsphere complex thereon. The digoxin-specific antibody-fluorescent microsphere complex is preferably obtained by conjugation between a digoxin monoclonal antibody or a digoxin polyclonal antibody and a fluorescent microsphere. The mass percentage concentration of the digoxin-specific antibody-fluorescent microsphere complex is preferably 0.01-0.1%; and the spraying amount of the digoxin-specific antibody-fluorescent microsphere complex on the glass fiber mat is preferably 1-10 μl/cm, more preferably 3-7 μl/cm, and most preferably 5 μl/cm.

The nitrocellulose membrane can be any nitrocellulose membrane as known by one of ordinary skill in the art, and a commercially available product of a conventionally-prepared test strip can be used. In the invention, the nitrocellulose membrane is sequentially provided with a detection line coated with digoxin protein conjugate and a quality control line coated with a secondary antibody. The digoxin protein conjugate preferably includes a digoxin-conjugated rabbit albumin or a digoxin-conjugated rabbit ovalbumin. The concentration of the digoxin protein conjugate is preferably 1-3 mg/mL. The spraying amount of the digoxin protein conjugate on the nitrocellulose membrane is preferably 1-5 μl/cm, more preferably 2-4 μl/cm, and most preferably 5 μl/cm. The secondary antibody preferably includes a goat anti-mouse IgG antibody; and the concentration of the goat anti-mouse IgG antibody is preferably 1-3 mg/mL.

The invention also provides the use of the immunochromatographic test strip described above in preparing a kit for detecting the digoxin content in a sample. It preferably includes the steps of 1) adding a sample to be tested dropwise onto a sample pad for chromatography; 2) reading fluorescence signal values, i.e., a T value and a C value, for the detection line and the quality control line of the immunochromatographic test strip after the chromatography; and 3) substituting the fluorescence signal values T/C into a standard linear regression equation y=ax³+bx²+cx+d to calculate the digoxin content in the sample to be tested.

In the invention, the test sample preferably includes serum, plasma, or whole blood.

The following clearly and completely describes the invention with reference to the preferred embodiments of the invention. The described embodiments are merely a part rather than all of the embodiments of the invention. All other embodiments obtained by a person of ordinary skill in the art based on the embodiments of the invention without creative efforts shall fall within the protection scope of the invention.

### Example 1

Preparation of Immunochromatographic Test Strip:

(1) Labeling Process:

A. fluorescent microsphere labeling: the fluorescent microspheres were commercially purchased from common fluorescent microspheres at a particle size of 80 nm. 1% fluorescent microspheres, 10 mg/ml of EDC, and 100 μg/ml of a digoxin monoclonal antibody were added, mixed well for conjugation for 3 h, centrifuged with the supernatant being removed, and then added with 1% BSA for blocking for 1 h.

B. colloidal gold labeling: colloidal gold was a colloidal gold solution with a particle size of 10 nm as prepared by a general-purpose trisodium citrate reduction method. 100 μg/ml of the digoxin monoclonal antibody was added to the colloidal gold solution, mixed well for conjugation for 3 h, centrifuged with the supernatant being removed, and then added with 1% BSA for blocking for 1 h.

(2) Gold Spraying Process:

the prepared labeled conjugate was centrifuged, and resuspended in a gold spraying buffer to a concentration of 0.2% for gold spraying, where

A. the resuspension buffer contains 50 mM buffer (tris, phosphoric acid, glycine, HEPES, PH=6.5), 1 g/L of sucrose, 3 g/L of polyethylene glycol 6000, 0.1 g/L of glycine, 0.05 g/L of arginine, and 3 g/L of mannitol;

B. pre-treating a gold cushion before gold spraying was necessary, wherein the treatment buffer contains 50 mM buffer (tris, phosphoric acid, glycine, HEPES, PH=6.5, being consistent with the gold spraying buffer), 0.1% txiton-100, and 3 g/L of mannitol;

C. the gold spraying equipment used was a gold spray membrane scribing meter; and

D. after the gold spraying was completed, the product was baked in a 30° C. air drying oven for drying for 6 h;

(3) Membrane Scribing Process:

A. the digoxin protein conjugate was a digoxin-conjugated rabbit albumin, at a concentration of 1 mg/ml;

B. the independent control line was goat anti-mouse IgG, at a concentration of 1 mg/ml;

C. membrane scribing is conducted for the conjugate and the quality control line respectively on a T line position and a C line position of the nitrocellulose membrane; and

D. after the membrane scribing was completed, the product was baked in a 30° C. air drying oven for drying for 4 h;

(4) Sample Pad Treatment Process:

the sample pad was soaked in the 0.1% txiton-100 for 2 h to promote sample release;

(5) Board Pasting Process:

this process was a general process, in which the sample pad, the dried gold standard pad, the dried nitrocellulose film, and the absorbent paper were sequentially adhered to the bottom board; and

(6) Strip Cutting and Packing Process:

this process was a general process, in which a large reagent plate was cut, then loaded into a reagent card, added with a desiccant and sealed with an aluminum foil bag to obtain the test strip.

### Example 2

Preparation of Immunochromatographic Test Strip:

(1) Labeling Process:

A. fluorescent microsphere labeling: the fluorescent microspheres were commercially purchased from common fluorescent microspheres at a particle size of 120 nm. 1% fluorescent microspheres, 10 mg/ml of EDC, and 500 μg/ml of a digoxin polyclonal antibody were added to the fluorescent microspheres, mixed well for conjugation for 3 h, centrifuged with the supernatant being removed, and then added with 1% BSA for blocking for 1 h.

B. colloidal gold labeling: colloidal gold was a colloidal gold solution with a particle size of 50 nm as prepared by a general-purpose trisodium citrate reduction method. 500 μg/ml of the digoxin polyclonal antibody was added to the colloidal gold solution, mixed well for conjugation for 3 h, centrifuged with the supernatant being removed, and then added with 1% BSA for blocking for 1 h;

(2) Gold Spraying Process:

the prepared labeled conjugate was centrifuged, and resuspended in a gold spraying buffer to a concentration of 0.2% for gold spraying, where

A. the resuspension buffer contains 200 mM buffer (tris, phosphoric acid, glycine, HEPES, PH=7.8), 2 g/L of sucrose, 10 g/L of polyethylene glycol 6000, 1 g/L of glycine, 0.5 g/L of arginine, and 15 g/L of mannitol;

B. pre-treating a gold cushion before gold spraying was necessary, wherein the treatment buffer contains 200 mM buffer (tris, phosphoric acid, glycine, HEPES, PH=7.8, being consistent with the gold spraying buffer), 0.1% txiton-100, and 15 g/L of mannitol;

C. the gold spraying equipment used was a gold spray membrane scribing meter; and

D. after the gold spraying was completed, the product was baked in a 40° C. air drying oven for drying for 10 h;

(3) Membrane Scribing Process:

A. the digoxin protein conjugate was a digoxin-conjugated rabbit ovalbumin, at a concentration of 3 mg/ml;

B. an independent control line was goat anti-mouse IgG, at a concentration of 3 mg/ml;

C. membrane scribing was conducted for the conjugate and the quality control line respectively on a T line position and a C line position of the nitrocellulose membrane; and

D. after the membrane scribing was completed, the product was baked in a 40° C. air drying oven for drying for 10 h;

(4) Sample Pad Treatment Process:

the sample pad was soaked in the 0.1% txiton-100 for 2 h to promote sample release;

(5) Board Pasting Process:

this process was a general process, in which the sample pad, the dried gold standard pad, the dried nitrocellulose film, and the absorbent paper were sequentially adhered to the bottom board; and

(6) Strip Cutting and Packing Process:

this process was a general process, in which a large reagent plate was cut, then loaded into a reagent card, added with a desiccant, and sealed with an aluminum foil bag to obtain the test strip.

### Example 3

Preparation of Immunochromatographic Reagent Strip:

(1) fluorescent microspheres with a particle size of 80 nm were used; where 1% fluorescent microspheres, 10 mg/ml of EDC, 200 μg/ml of the digoxin monoclonal antibody were added, mixed well for conjugation for 3 h, centrifuged with the supernatant being removed, and then added with 1% BSA for blocking for 1 h;

(2) the prepared labeling conjugate was centrifuged, and resuspended to 0.2% by using a resuspension buffer (a 200 mM glycine buffer, PH=6.5), 1 g/L of sucrose, 10 g/L of polyethylene glycol 6000, 1 g/L of glycine, 0.5 g/L of arginine, and 15 g/L of mannitol; the gold cushion was soaked in a pretreatment buffer (a 200 mM glycine buffer, 0.1% txiton-100, and 15 g/L of mannitol) for 2 h, baked for 1 h; the resuspended solution was sprayed onto the gold cushion to form a membrane by using a gold spray membrane scribing meter; and after the gold spraying was completed, the product was baked in a 30° C. air drying oven for drying for 10 h;

(3) the T line was a digoxin-conjugated BSA at a concentration of 1 mg/ml; the C line was a goat anti-mouse IgG at a concentration of 1 mg/ml; membrane scribing was conducted for the conjugate and the quality control line respectively on a T line position and a C line position of the nitrocellulose membrane, and after the membrane scribing was completed, the product was baked in a 40° C. air drying oven for drying for 4 h;

(4) sample pad treatment process: the sample pad was soaked in the 0.1% txiton-100 for 2 h to promote sample release; and

(5) the sample pad, the dried gold standard pad, the dried nitrocellulose membrane and the absorbent paper were sequentially adhered onto the bottom plate; the large reagent plate was cut, then loaded into the reagent card, added with a desiccant and sealed with an aluminum foil bag to obtain the test strip.

### Example 4

Preparation of Calibration Curve:

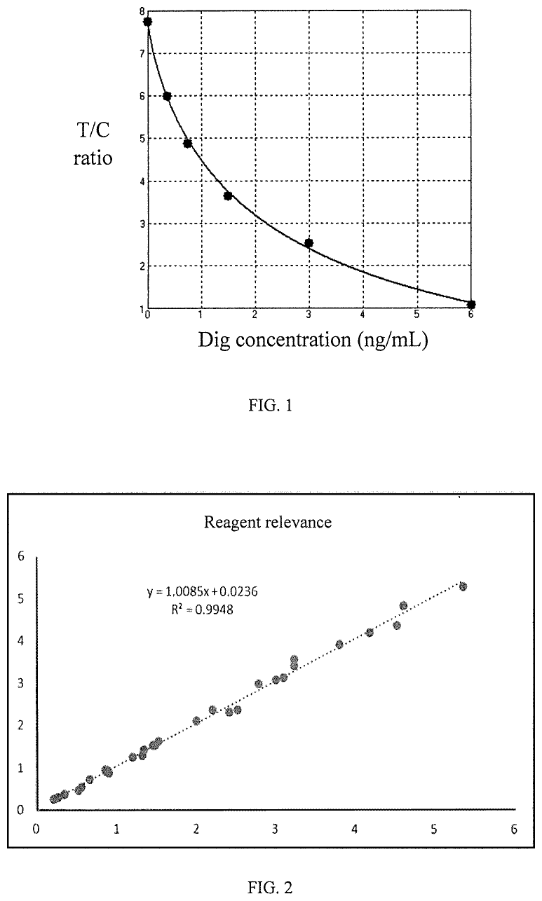

Digoxin calibrators at concentrations respectively of 0, 0.3125, 0.625, 1.25, 2.5, and 5 ng/ml were added dropwise onto the test strip of Example 3, and 3 replicates were set for each concentration. After the test strip was mixed well and subjected to static chromatography for 15 min, the fluorescence signal value was read using an immunofluorescence analyzer, and so as to calculate a T/C value and establish a calibration curve, where the X-axis is the concentration of the calibrator, and the Y-axis is the T/C value, as shown in FIG. 1.

Detection of Sample Repeatability:

A test sample was added dropwise into a sample loading well, and 10 replicates were set for each sample. The test sample was a serum sample, and no high-value natural sample could be obtained due to the metabolism process on digoxin, so the high-value sample was obtained by adding pure digoxin product into a clinical serum sample. The specific data is shown in Table 1:

TABLE 1
Detection of Sample Repeatability

| | Sample concentration (ug/ml) | | | | | | | | | | | | |
|---|---|---|---|---|---|---|---|---|---|---|---|---|---|
| | Deter- | Deter- | Deter- | Deter- | Deter- | Deter- | Deter- | Deter- | Deter- | Deter- | | | |
| Sample | mina- | mina- | mina- | mina- | mina- | mina- | mina- | mina- | mina- | mina- | Average | Standard | |
| No. | tio [symbol] | tio [symbol] | tio [symbol] | tio [symbol] | tio [symbol] | tio [symbol] | tio [symbol] | tio [symbol] | tio [symbol] | tio [symbol] | value | deviation | CV |
| 1 | 0.33 | 0.37 | 0.41 | 0.35 | 0.36 | 0.39 | 0.42 | 0.31 | 0.37 | 0.36 | 0.367 | 0.033682 | 9.18% |
| 2 | 0.49 | 0.52 | 0.46 | 0.43 | 0.52 | 0.55 | 0.53 | 0.51 | 0.48 | 0.51 | 0.5 | 0.03559 | 7.12% |
| 3 | 0.61 | 0.63 | 0.67 | 0.72 | 0.65 | 0.71 | 0.62 | 0.63 | 0.61 | 0.67 | 0.652 | 0.039665 | 6.08% |
| 4 | 0.72 | 0.82 | 0.73 | 0.81 | 0.69 | 0.76 | 0.72 | 0.75 | 0.79 | 0.83 | 0.762 | 0.048259 | 6.33% |
| 5 | 0.85 | 0.83 | 0.72 | 0.87 | 0.89 | 0.73 | 0.82 | 0.85 | 0.81 | 0.79 | 0.816 | 0.056016 | 6.86% |
| 6 | 0.91 | 0.93 | 0.91 | 1.03 | 0.95 | 1.05 | 1.02 | 0.93 | 1.04 | 1.02 | 0.979 | 0.057629 | 5.89% |
| 7 | 1.23 | 1.19 | 1.12 | 1.28 | 1.17 | 1.24 | 1.19 | 1.27 | 1.22 | 1.18 | 1.21625 | 0.052082 | 4.28% |
| 8 | 2.01 | 2.12 | 2.17 | 2.2 | 2.08 | 2.02 | 2.19 | 2.09 | 2.12 | 2.04 | 2.104 | 0.068508 | 3.26% |
| 9 | 3.21 | 3.29 | 3.08 | 3.19 | 3.03 | 3.21 | 3.15 | 3.03 | 3.23 | 3.03 | 3.145 | 0.09583 | 3.05% |
| 10 | 4.83 | 4.53 | 4.95 | 4.79 | 4.81 | 4.65 | 4.88 | 4.79 | 4.52 | 4.83 | 4.758 | 0.144207 | 3.03% |

[symbol]

indicates data missing or illegible when filed

It can be seen from Table 1 that, the samples with different concentrations in the whole linear range were tested, and all of the CVs of the detection results were less than 10%, with good repeatability, which satisfied the test requirements.

### Example 5

Detection of Anti-Interference Property

For routine blood detection, 50 mg/ml hemoglobin and 50 mg/dl triglyceride were added to the test sample. The values before and after the addition were tested to observe whether a conventional clinical interference substance has an effect on the determination results. Table 2 shows the results:

TABLE 2
Effect of interfering substance on determination results

| | Before the addition of interfering substance | | | | After the addition of interfering substance | | | | |
|---|---|---|---|---|---|---|---|---|---|
| Sample | | | | Average | | | | Average | Relative |
| No. | Determinati [symbol] | Determinati [symbol] | Determinati [symbol] | value | Determinati [symbol] | Determinati [symbol] | Determinati [symbol] | [symbol] | deviation |
| 1 | 0.31 | 0.33 | 0.3 | 0.313333333 | 0.34 | 0.33 | 0.35 | 0.31 | −1.06% |
| 2 | 0.72 | 0.75 | 0.73 | 0.733333333 | 0.74 | 0.75 | 0.76 | 0.75 | 2.27% |
| 3 | 1.37 | 1.54 | 1.42 | 1.443333333 | 1.36 | 1.51 | 1.42 | 1.43 | −0.92% |
| 4 | 1.89 | 1.82 | 1.67 | 1.793333333 | 1.76 | 1.62 | 1.81 | 1.73 | −3.53% |

[symbol]

indicates data missing or illegible when filed

It can be seen from Table 2 that, the relative deviation before and after the addition of the interfering substance was less than 5%, which was an acceptable range.

### Example 6

Comparison of Whole Blood Sample Detection Results with Serum Sample Detection Results:

The whole blood sample selected in this experiment was from the same source as the serum sample. The reliability of the detection of the whole blood sample by this reagent was determined by comparing the serum detection results and the whole blood detection results, as shown in Table 3.

TABLE 3
Detection results of control serum and whole blood

| | Determined value of serum | | | | Determined value of whole blood | | | | |
|---|---|---|---|---|---|---|---|---|---|
| Sample | | | | Average | | | | Average | Relative |
| No. | Determin [symbol] | Determin [symbol] | Determin [symbol] | value | Determin [symbol] | Determin [symbol] | Determin [symbol] | value | deviation |
| 1 | 1.45 | 1.32 | 1.36 | 1.376666667 | 1.36 | 1.35 | 1.51 | 1.406667 | 2.18% |
| 2 | 1.67 | 1.73 | 1.82 | 1.74 | 1.65 | 1.83 | 1.72 | 1.733333 | −0.38% |
| 3 | 1.37 | 1.54 | 1.42 | 1.443333333 | 1.41 | 1.43 | 1.5 | 1.446667 | 0.23% |
| 4 | 1.89 | 1.82 | 1.67 | 1.793333333 | 1.84 | 1.76 | 1.72 | 1.773333 | −1.12% |
| 5 | 0.58 | 0.61 | 0.68 | 0.623333333 | 0.58 | 0.67 | 0.65 | 0.633333 | 1.60% |
| 6 | 0.74 | 0.65 | 0.72 | 0.703333333 | 0.68 | 0.78 | 0.72 | 0.726667 | 3.32% |
| 7 | 1.05 | 1.03 | 1.02 | 1.033333333 | 1.05 | 0.99 | 1.03 | 1.023333 | −0.97% |
| 8 | 1.13 | 1.24 | 1.15 | 1.173333333 | 1.07 | 1.24 | 1.32 | 1.21 | 3.13% |
| 9 | 2.32 | 2.24 | 2.36 | 2.306666667 | 2.31 | 2.27 | 2.41 | 2.33 | 1.01% |
| 10 | 1.32 | 1.41 | 1.37 | 1.366666667 | 1.35 | 1.32 | 1.34 | 1.336667 | −2.20% |

[symbol]

indicates data missing or illegible when filed

It can be seen from Table 3 that, the deviation between the whole blood sample and the serum sample was within 5%, which was acceptable.

### Comparative Example 1

Comparison of stability: the shelf life of the test strip from Example 3 of the invention was 18 months when stored at room temperature 2-30° C., and the shelf life of a control reagent, i.e., a digoxin determination kit (legal luminescent microparticle immunoassay) produced by Abbott, was 18 months when stored at 2-8° C. The test strip of the invention was superior to the control kit in terms of the storage temperature.

Clinical relevance: the type of the test sample for the test strip of the invention may be serum, plasma, or whole blood, and the type of the test sample for the control reagent did not include the whole blood. Whole blood could not be directly loaded onto a machine due to the presence of blood cells. Therefore, the type of the control sample was serum, and the detection result of the whole blood sample was shown in Example 6. The control reagent was a digoxin determination kit (legal luminescent microparticle immunoassay) produced by Abbott. For the results, see Table 4 and FIG. 2.

TABLE 4
Test reagent and control reagent of the invention

| Sample No. | Test reagent | Control reagent |
|---|---|---|
| 1 | 0.22 | 0.25 |
| 2 | 0.35 | 0.37 |
| 3 | 0.67 | 0.71 |
| 4 | 0.27 | 0.29 |
| 5 | 1.35 | 1.41 |
| 6 | 0.87 | 0.91 |
| 7 | 1.49 | 1.52 |
| 8 | 2.21 | 2.36 |
| 9 | 1.33 | 1.28 |
| 10 | 2.53 | 2.36 |
| 11 | 2.42 | 2.31 |
| 12 | 5.36 | 5.28 |
| 13 | 4.61 | 4.83 |
| 14 | 0.56 | 0.53 |
| 15 | 0.89 | 0.93 |
| 16 | 1.46 | 1.53 |
| 17 | 0.53 | 0.46 |
| 18 | 0.86 | 0.95 |
| 19 | 1.53 | 1.62 |
| 20 | 2.01 | 2.11 |
| 21 | 4.53 | 4.37 |
| 22 | 3.24 | 3.41 |
| 23 | 3.81 | 3.92 |
| 24 | 0.91 | 0.87 |
| 25 | 1.21 | 1.25 |
| 26 | 3.01 | 3.08 |
| 27 | 3.11 | 3.13 |
| 28 | 4.19 | 4.21 |
| 29 | 3.24 | 3.56 |
| 30 | 2.79 | 2.98 |

As can be seen from Table 4, the clinical relevance is greater than 0.9, which is in line with clinical use requirements.

It can be seen from the results of the above examples that, the immunochromatographic test strip provided by the invention has good repeatability and an anti-interference property within an acceptable range, can be used for detecting the whole blood sample, and has excellent stability.

The foregoing descriptions are only preferred embodiments of the invention. It should be noted that for a person of ordinary skill in the art, several improvements and modifications may further be made without departing from the principle of the invention. These improvements and modifications should also be deemed as falling within the protection scope of the invention.

1. An immunochromatographic test strip for detecting digoxin, comprising a bottom plate, and a sample pad, a glass fiber mat, a nitrocellulose membrane and a water absorbing paper which are sequentially overlapped on the bottom plate, wherein the glass fiber mat is sprayed with a digoxin-specific antibody-fluorescent microsphere complex; and the nitrocellulose membrane is sequentially provided with a detection line coated with digoxin protein conjugate and a quality control line coated with a secondary antibody;
the digoxin-specific antibody-fluorescent microsphere complex is obtained by conjugation between a digoxin monoclonal antibody or a digoxin polyclonal antibody and a fluorescent microsphere; and
the digoxin protein conjugate comprises a digoxin-conjugated rabbit albumin or a digoxin-conjugated rabbit ovalbumin;
the mass percentage concentration of the digoxin-specific antibody-fluorescent microsphere complex is 0.01-0.1%; and the spraying amount of the digoxin-specific antibody-fluorescent microsphere complex on the glass fiber mat is 1-10 μl/cm;
the test sample comprises serum, plasma and whole blood.

2. (canceled)

3. The immunochromatographic test strip for detecting digoxin according to claim 1, wherein the concentration of the digoxin protein conjugate is 1-3 mg/mL; and the spraying amount of the digoxin protein conjugate on the nitrocellulose membrane is 1-5 μl/cm.

4. (canceled)

5. The immunochromatographic test strip for detecting digoxin according to claim 1, wherein the secondary antibody comprises a goat anti-mouse IgG antibody.

6. (canceled)

7. The immunochromatographic test strip for detecting digoxin according to claim 5, wherein the concentration of the goat anti-mouse IgG antibody is 1-3 mg/mL.

8. (canceled)

9. A use of the immunochromatographic test strip for detecting digoxin according to claim 1 in preparing a kit for detecting the digoxin content in a sample.

10. (canceled)

11. A use of the immunochromatographic test strip for detecting digoxin according to claim 3 in preparing a kit for detecting the digoxin content in a sample.

12. (canceled)

13. A use of the immunochromatographic test strip for detecting digoxin according to claim 5 in preparing a kit for detecting the digoxin content in a sample.

14. (canceled)

15. A use of the immunochromatographic test strip for detecting digoxin according to claim 7 in preparing a kit for detecting the digoxin content in a sample.

16. (canceled)

17. The use according to claim 9, wherein the detecting the digoxin content in the sample comprises the steps of:
1) adding a sample to be tested dropwise onto a sample pad for chromatography;
2) reading fluorescence signal values, for the detection line and the quality control line of the immunochromatographic test strip after the chromatography; and
3) substituting the fluorescence signal values T/C into a standard linear regression equation to calculate the digoxin content in the sample to be tested.

18. (canceled)

19. (canceled)

20. (canceled)
